(19) United States
(12) Patent Application Publication
(10) Pub. No.: US 2026/0335444 A1
(43) Pub. Date: Oct. 29, 2026

(54) ORGANICALLY FUNCTIONALIZED INORGANIC MATERIAL AND CORRESPONDING METHOD

(71) Applicant: KING ABDULLAH UNIVERSITY OF SCIENCE AND TECHNOLOGY, Thuwal (SA)

(72) Inventors: Anissa BENDJERIOU-SEDJERARI, Thuwal (SA); Swechchha PANDEY, Thuwal (SA); Magnus RUEPING, Thuwal (SA)

(21) Appl. No.: 19/652,376

(22) Filed: Apr. 20, 2026

Related U.S. Application Data
(60) Provisional application No. 63/796,597, filed on Apr. 29, 2025.

(51) Int. Cl. A61K 47/52; A61K 47/54

(52) U.S. Cl. CPC A61K 47/52; A61K 47/545

(57) ABSTRACT

A linker free, rigid, organic-inorganic hybrid composition includes a silica stripped of an NH₂ group, an organic compound, and a single oxygen atom directly linking the silica stripped of the NH₂ group to the organic compound.

### CROSS-REFERENCE TO RELATED APPLICATIONS

This application claims priority to U.S. Provisional Patent Application No. 63/796,597, filed on Apr. 29, 2025, entitled “ORGANICALLY FUNCTIONALIZED INORGANIC MATERIAL AND CORRESPONDING METHOD,” the disclosure of which is incorporated herein by reference in its entirety.

### BACKGROUND OF THE INVENTION

### Technical Field

Embodiments of the subject matter disclosed herein generally relate to an organic-inorganic hybrid composition and a method for making the composition, and more particularly, to a composition that includes a linker free, organic-inorganic hybrid material.

### Discussion of the Background

The development of single materials that combine the properties of organic and inorganic building blocks, known as hybrid materials, is particularly attractive from the perspective of materials scientists. Indeed, these types of functionalized materials feature a wide range of benefits from the variation of organic moieties to thermal and mechanical stability of inorganic substrates, which are particularly demanding in various applications such as, but not limited to, heterogeneous catalysis. To date, organic-inorganic hybrid materials, such as nonporous silica, silica-alumina, mesoporous silica, fibrous silica, zeolites, fibrous hierarchical zeolite, mesoporous zeolite, etc. were developed.

For example, the mesoporous silica is a highly versatile material characterized by its well-ordered porous structure with pore diameters ranging from 2 to 50 nanometers. Due to its unique properties, such as high surface area, tunable pore size, and chemical stability, mesoporous silica has garnered significant attention across various scientific and industrial fields. Its ability to be functionalized with different chemical groups further enhances its applicability in numerous areas.

One advantage of the mesoporous silica lies in its structural and chemical properties, which enable it to function effectively in various applications. Some of its applications include catalysis support, drug delivery substance, adsorption and separation function, energy storage and conversion, biosensing and medical applications.

When used for catalysis support, the mesoporous silica serves as an excellent support material for catalysts due to its large surface area and uniform pore distribution. It provides an environment where catalytic reactions can occur efficiently, improving the reactivity and selectivity of catalysts. Mesoporous silica is frequently employed as a catalyst support in heterogeneous catalysis. The well-defined pore structure provides a suitable environment for catalysts such as metal nanoparticles and enzymes, improving reaction efficiency. For example, in petrochemical refining and fine chemical synthesis, mesoporous silica-based catalysts enhance reaction selectivity and reduce energy consumption.

For the drug delivery application, due to its biocompatibility and tunable pore size, the mesoporous silica is widely used in controlled drug delivery. It allows for the encapsulation of drugs and their sustained release, ensuring improved therapeutic effects while minimizing side effects. The porous structure of this material allows drugs to be loaded into the silica framework and released in a controlled manner. This is particularly beneficial for cancer therapy, where targeted drug release can improve treatment outcomes while minimizing side effects. Functionalized mesoporous silica nanoparticles (MSNs) are also being explored for gene therapy and vaccine delivery.

Regarding the adsorption and separation application, the mesoporous silica is highly effective in adsorbing pollutants, organic compounds, and heavy metals from wastewater and air. Its adjustable surface chemistry enables selective adsorption and separation processes. Industries such as gas purification, chromatography, and food processing benefit from mesoporous silica's selective adsorption capabilities. In gas separation, it is used to selectively adsorb CO₂, nitrogen, and other gases, improving energy efficiency in industrial processes. Furthermore, mesoporous silica is widely used in chromatography to separate and analyze complex mixtures in pharmaceuticals and biochemical research. Mesoporous silica is also used in environmental remediation processes, including water purification and air filtration. Its ability to adsorb pollutants such as heavy metals (lead, arsenic) and organic contaminants (pesticides, dyes) makes it an effective material for environmental cleanup. Additionally, mesoporous silica is employed in carbon capture technologies to reduce greenhouse gas emissions.

With regard to the energy storage and conversion function, this material is used in supercapacitors and lithium-ion batteries to enhance performance by providing high conductivity and efficient ion transport. Mesoporous silica materials are integrated into energy storage systems to enhance battery and supercapacitor performance. The high surface area and well-organized pore structure contribute to improved charge storage, faster ion transport, and higher energy densities. This makes mesoporous silica a promising material for next-generation energy storage devices.

Finally, with regard to the biosensing and medical applications, the mesoporous silica plays a role in developing nanostructured sensors for detecting biomolecules and pathogens. Advancements in nanotechnology have enabled the use of mesoporous silica in biosensors for detecting pathogens, proteins, and small molecules with high sensitivity. Additionally, MSNs have been explored for medical imaging applications, where they can serve as contrast agents for magnetic resonance imaging (MRI) and fluorescence imaging. While the above description explores the advantages of mesoporous silica, similar advantages are shown by other organic-inorganic hybrid materials.

However, the existing methods for making the organic-inorganic hybrid materials are not environmentally friendly, they are time-consuming, and their large-scale production is limited. Also, the existing organic-inorganic hybrid materials include or require the use of materials that are damaging to the environment. Thus, there is a need for a new method for making such materials and also for new organic-inorganic hybrid materials that do not require and/or do not include toxic materials.

### SUMMARY OF THE INVENTION

According to an embodiment, there is a linker free, rigid, organic-inorganic hybrid composition that includes a silica stripped of an NH₂ group, an organic compound, and a single oxygen atom directly linking the silica stripped of the NH₂ group to the organic compound.

According to another embodiment, there is a method for making a linker free, rigid, organic-inorganic hybrid composition, and the method includes a step of providing a silica having an NH₂ group, a step of providing an organic compound, and a step of reacting the silica with the NH₂ group with the organic compound so that a single oxygen atom directly links a silica stripped of the NH₂ group to the organic compound to form the linker free, rigid, organic-inorganic hybrid composition.

### BRIEF DESCRIPTION OF THE DRAWINGS

For a more complete understanding of the present invention, reference is now made to the following descriptions taken in conjunction with the accompanying drawings, in which:

FIG. 1 is a schematic diagram of a sol-gel co-condensation reaction that uses an organoalkoxysilane;

FIG. 2 is a schematic diagram of a post-synthetic functionalization reaction that uses an organoalkoxysilane;

FIGS. 3A and 3B are schematic diagrams of a post-synthetic functionalization reaction that uses Grignard reagents or Chlorosilane;

FIG. 4 is a schematic diagram of a sol-gel co-condensation method that generates a TEMPO-functionalized material using an organoalkoxysilane;

FIG. 5 is a schematic diagram of a post-synthesis method that generates TEMPO-functionalized materials using an organoalkoxysilane;

FIGS. 6A to 6C are schematic diagrams illustrating mesoporous silica surface modification using 4-hydroxy-TEMPO, carboxy-TEMPO, and its succinimide derivative, respectively;

FIG. 7 illustrates a method for the generation of a carboxylic acid functionalized polymer;

FIG. 8 illustrates a single step method for generating a linker free, rigid, TEMPO-functionalized silica composition without the use of organoalkoxysilane;

FIG. 9 is a flow chart of the method for generating the linker free, rigid, TEMPO-functionalized silica composition without the use of organoalkoxysilane illustrated in FIG. 8;

FIG. 10 compares the linker free, rigid, TEMPO-functionalized silica of FIG. 8 with a traditional TEMPO-functionalized silica that includes an alkyl linker;

FIG. 11 shows the Fourier Transform Infrared Spectroscopy spectra of a silica surface having a silylamine surface group and a silica after a reaction with 4-hydroxyl TEMPO to lead to the linker free, rigid, TEMPO-functionalized silica of FIG. 8;

FIG. 12 illustrates the electron paramagnetic resonance spectrum of the linker free, rigid, TEMPO-functionalized silica of FIG. 8;

FIG. 13 illustrates the thermogravimetric analysis curve of the linker free, rigid, TEMPO-functionalized silica of FIG. 8;

FIG. 14 illustrates a single step method for generating a linker free, rigid, TOTAPOL-functionalized silica composition without the use of organoalkoxysilane;

FIG. 15 illustrates an oxidation reaction catalyzed by the linker free, rigid, TEMPO-functionalized silica composition of FIG. 8;

FIG. 16 illustrates a table that includes various parameters of the linker free, rigid, TEMPO-functionalized silica of FIG. 8 and the linker free, rigid, TOTAPOL-functionalized silica composition of FIG. 14;

FIGS. 17A and 17B illustrate the ¹³C relaxation time in pyruvic acid in the presence of TEMPO functionalized silica when the microwave is on vs when the microwave is off in dynamic nuclear polarization (DNP) experimental conditions;

FIG. 18 illustrates a table with possible organic compounds for the linker free, rigid, organic-functionalized silica composition of FIGS. 8 and 14; and

FIG. 19 illustrates a single step method for generating a linker free, rigid, organically functionalized silica composition without the use of organoalkoxysilane.

### DETAILED DESCRIPTION OF THE INVENTION

The following description of the embodiments refers to the accompanying drawings. The same reference numbers in different drawings identify the same or similar elements. The following detailed description does not limit the invention. In-stead, the scope of the invention is defined by the appended claims. The following embodiments are discussed, for simplicity, with regard to the functionalization of silica with 4-hydroxy-TEMPO and its TOTAPOL derivatives. However, the embodiments to be discussed next are not limited to such materials but may be applied to other sup-porting materials functionalized with other organic compounds.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

According to an embodiment, a novel and cost-effective methodology to manufacture functionalized materials (hybrid organic-inorganic materials) is disclosed. Various catalysts generated with this method include the functionalization of silica with 4-hydroxy-TEMPO and its TOTAPOL derivatives.

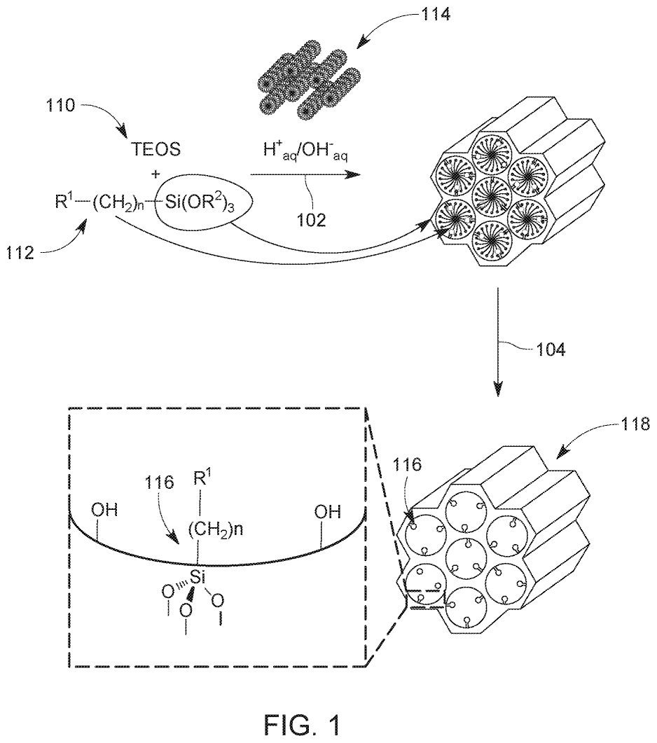

There are methodologies currently used for making the above discussed organic-inorganic hybrid materials, for example, the sol-gel co-condensation method and the post-synthetic functionalization. The sol-gel co-condensation method consists of a simultaneous condensation 102, as illustrated in FIG. 1, of an organosilica precursor 110, e.g., tetraethyl orthosilicate (TEOS) or tetramethyl orthosilicate (TMOS), and a given organoalkoxysilane 112 of the type R¹—(CH₂)ₙSi(OR²)₃, with R¹ being an organic group such as —NH₂, —Cl, —N₃, —COOH, etc., R² being a methyl, ethyl group, and CH₂ aliphatic or cyclic compounds acting as a ‘spacer’ between the organic group and inorganic surface. The simultaneous condensation and hydrolysis take place in the presence of a template 114, which is used to grow the silica framework. The template 114 may be a surfactant. In one embodiment, the template is a cylindrical micelle that confers the mesostructure of the final hybrid material (for example, after calcination). The template is removed in step 104, by calcination, e.g., exposing the product to a high temperature, for example, about 550 °C. A Si—C bond 116 is obtained for the organic-inorganic hybrid material 118, as schematically illustrated in the figure.

The term “sol-gel” refers to the two stages of the process. The sol phase, which is the initial stage where the precursors (like metal alkoxides) are dispersed in a liquid, form a colloidal solution or “sol.” At this point, the particles are small and remain suspended in the liquid. As the process continues, the condensation reaction leads to the formation of a three-dimensional network. This network traps the liquid within it, resulting in a semi-solid material called a “gel.” The transition from a sol to a gel is the defining characteristic of this process, hence the name “sol-gel.” From there, further drying or heating can turn the gel into a solid material with tailored properties.

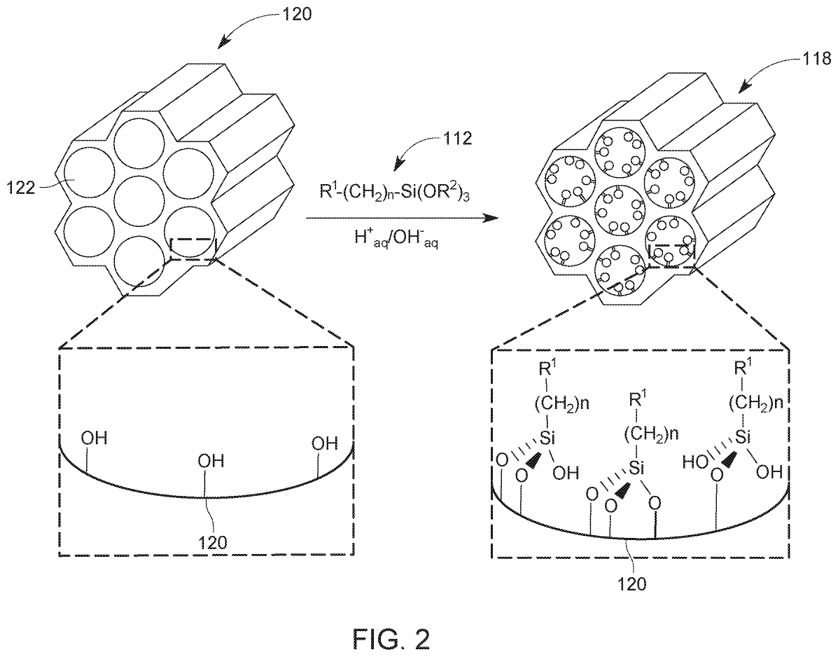

The post-synthetic functionalization method, which is schematically illustrated in FIG. 2, uses a subsequent modification of the surface of purely inorganic material by a given organoalkoxysilane of the type R¹—(CH₂)ₙSi(OR²)₃. More specifically, the mesoporous silica 120 having the mesopores 122, obtained by other means, is reacted with the organoalkoxysilane 112 to obtain the organic-inorganic hybrid material 118.

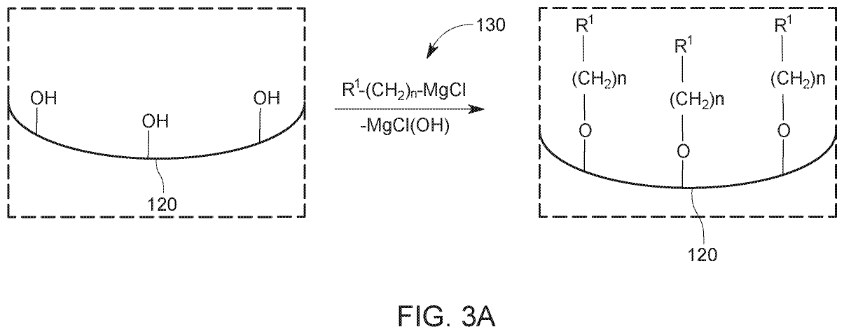

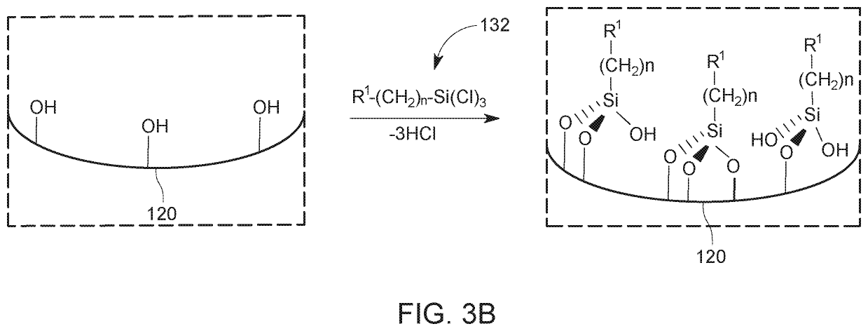

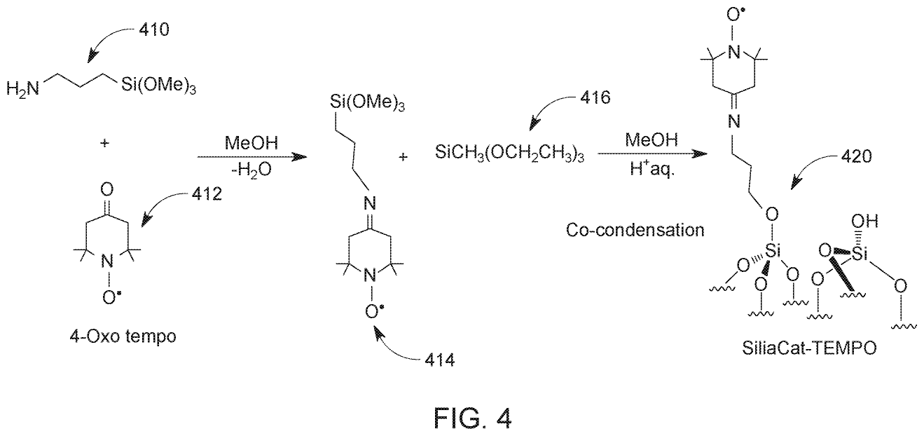

A variant of the method shown in FIG. 2 is the post-synthetic functionalization with Grignard reagents or chlorosilane. The organofunctionalization of silica by a post-synthetic approach can also involve a Grignard reagent 130, e.g., R₁(CH₂)ₙMgCl, or chlorosilane 132, e.g., R1(CH₂)ₙSiCl₃, as schematically illustrated in FIGS. 3A and 3B, respectively.

The organoalkoxysilanes discussed above with regard to FIGS. 1 and 2 are the most common substances used for the functionalization of oxide materials, followed by Grignard reagent and the chlorosilane. More recently, alkenylsilanes such as allyl-, methallyl-, vinyl-, and styryl silanes have been developed for introducing organic functional groups onto silica with the use of an acid catalyst [1, 2]. However, in all these cases, the manufacturing of the organic precursors and the functionalization process are still not eco-environmental friendly, they are still time-consuming, and their large-scale production is limited.

Although the organoalkoxysilanes are commercially available (e.g., aminopropyltriethoxysilan, 3-Mercapto-propyltriethoxysilane, (3-Iodopropyl)trimethoxysilane, (4-Chlorophenyl)triethoxysilane, (3-glycidyloxypropyl)trimethoxysilane, and 3-(Triethoxysilyl)propyl isocyanate), their industrial manufacture remain challenging as they require several synthesis and purification steps (i.e., chromatography, and crystallization) with the use of a large amount of toxic solvents.

Karsted's catalyst is used to synthesize these organoalkoxysilane which is highly expensive, hazardous and moisture sensitive.

In addition, the resulting organoalkoxysilanes are hazardous compounds and are highly sensitive to moisture as they can polymerize quickly. They also need to be manufactured and stored in a closed system under an inert atmosphere, which is not appropriate for large scale manufacturing. Further, the functionalization of the inorganic surfaces using organoalkoxysilane requires tedious and time-consuming synthetic steps. Indeed, further transformations of either the organoalkoxysilane itself or the functionalized materials are required, depending on the targeting applications.

In spite of the negative impact on the environment of the above hybrid materials and/or their manufacturing methods, the industry continues to produce new hybrid materials. For example, using the sol-gel co-condensation method, it is possible to manufacture TEMPO-functionalized over silica gel (porous or non-porous), known under the tradename SiliaCat® TEMPO with other SiliaCat® Heterogeneous Catalysts. Note that a TEMPO material or catalysis refers to a compound based on 2,2,6,6-tetramethylpiperidinyloxy (TEMPO), which is a stable nitroxyl radical. It is widely used in organic chemistry, particularly as a catalyst for oxidation reactions. TEMPO is highly effective in converting primary alcohols into aldehydes and secondary alcohols into ketones under mild and environmentally friendly conditions. One of its key advantages is its ability to promote selective oxidation without over-oxidizing the products. This makes it a popular choice in green chemistry applications. TEMPO can also be modified to enhance its reactivity or tailor it for specific reactions.

For this manufacturing process, 3-aminopropyltrimethoxysilane (NH₂—(CH₂)₃—Si(OMe)₃) 410, which provides the amine function, is mixed with a solution of 4-oxo-TEMPO 412 in methanol. Then, the resulting organoalkoxysilane 414, which is functionalized with a radical, is copolymerized with organosilanes 416, for example methyltriethoxysilane (MTES), according to the sol-gel co-condensation procedure. This reaction results in the SiliaCat-TEMPO 420.

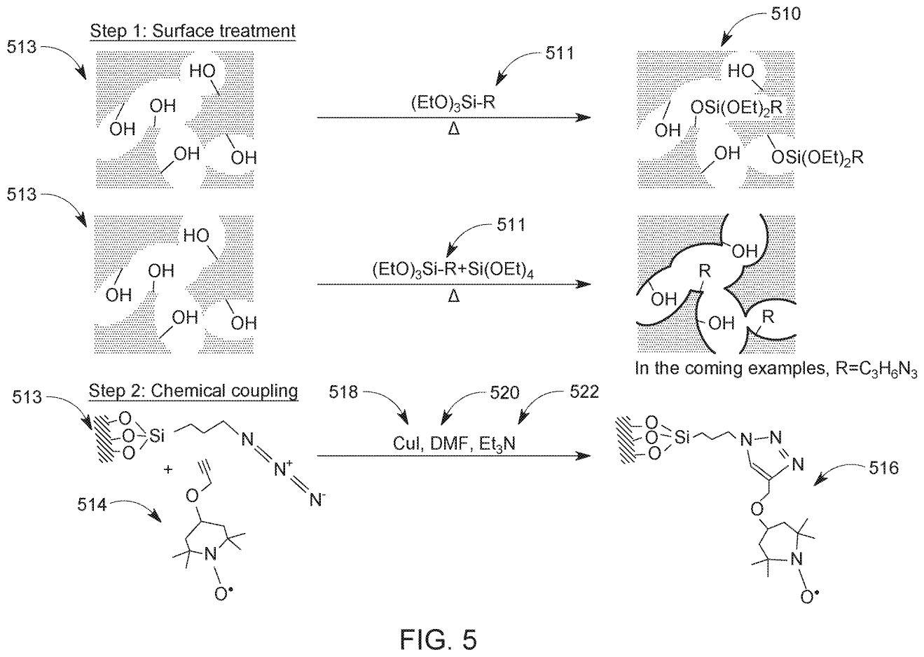

The post-synthesis method is also used to develop TEMPO-functionalized silica with different types of organoalkoxysilanes. For example, FIG. 5 illustrates a step 1 for the synthesis of 3-azido propyl-functionalized silica 510 followed by a step 2 involving a reaction between an azido group 512 and alkyne 514 for the generation of HYPSO 516. HYPSO stands for hyperpolarizing solids, which is obtained by the incorporation of a TEMPO radical 511 into a silica network 513. FIG. 5 shows two different reactants that provide the TEMPO radical 511. The starting reagents for the step 2 reaction may be Mat-Azide as the azido group 512, 4-propargylether TEMPO as the alkyne 514, and copper iodide 518, dimethylformamide 520, and triethylamine 522 as the catalysts.

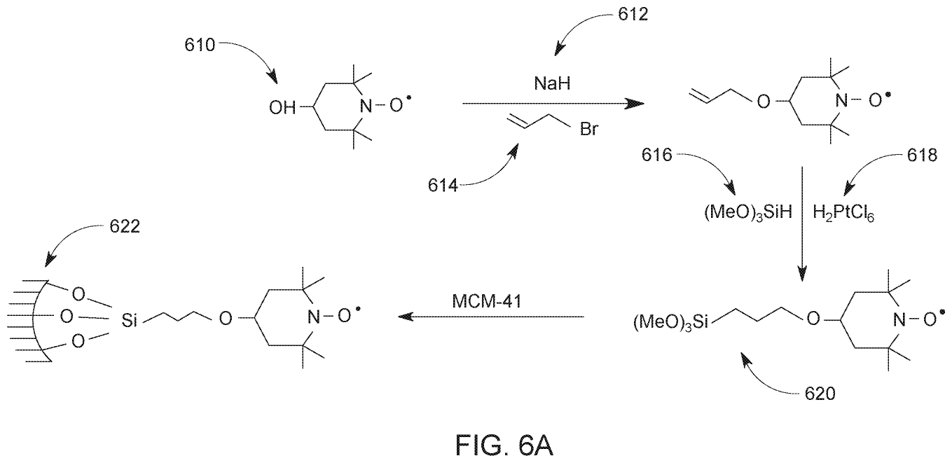

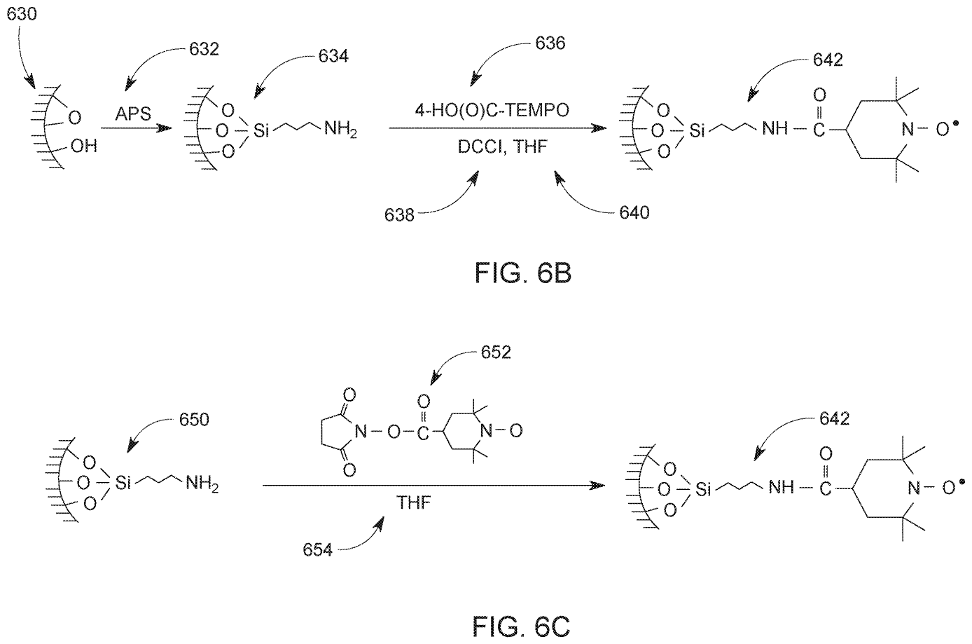

FIGS. 6A to 6C illustrate the modification of the mesoporous silica surface using 4-hydroxy-TEMPO (FIG. 6A), carboxy-TEMPO (FIG. 6B), and its succinimide derivative (FIG. 6C). More specifically, FIG. 6A shows the reaction of TEMPOL 610 with sodium hydride 612 and allyl bromide 614, which results in 4-allyloxy-TEMPO, which further interacts with trimethoxy silane 616 and chloroplatinic acid 618 to produce 3-(4-oxy-TEMPO)propyltrimethoxysilane 620, which eventually transforms into TEMPO-ether-MCM-41 622.

FIG. 6B shows how the MCM-41 silica 630 interacts with aminopropyl silane 632 to form 3-aminopropylsilyl chains 634, which in the presence of 4-carboxy TEMPO 636, N,N-dicyclohexyldicarbodiimide (DCCI) 638, and THF-Tetrahydrofuran 640, results in the generation of TEMPO-amide-MCM-41 642. FIG. 6C shows how the amine grafted MCM-41 silica 650 interacts with N-hydroxysuccinimide ester of 4-carboxy-TEMPO 652 in the presence of THF-Tetrahydrofuran 654 to form TEMPO-amide-MCM-41 642.

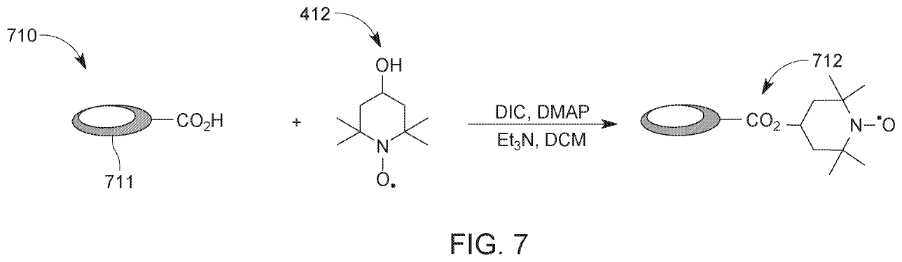

Another method for synthesizing a TEMPO based catalyst is illustrated in FIG. 7. A carboxylic acid fibreCat™ 710 interacts with TEMPO 412 and results in the TEMPO FibreCat™ 712, which is a nitroxyl radical version of the catalyst 710, i.e., a carboxylic acid functionalized polymer. Note that the catalyst 710 includes a Merrifield resin or a polymer support 711.

However, even these more modern methods and the resulting catalysts suffer from the problems of the methods discussed with regard to FIGS. 1 to 3B, i.e., they are still detrimental to the environment, are time consuming, and they are not suitable for large scale industrial applications. Thus, the inventors have developed new methods and new TEMPO-functionalized compositions (e.g., catalysts) that overcome one or more of the problems discussed above. These methods do not require the synthesis of expensive organoalkoxysilane.

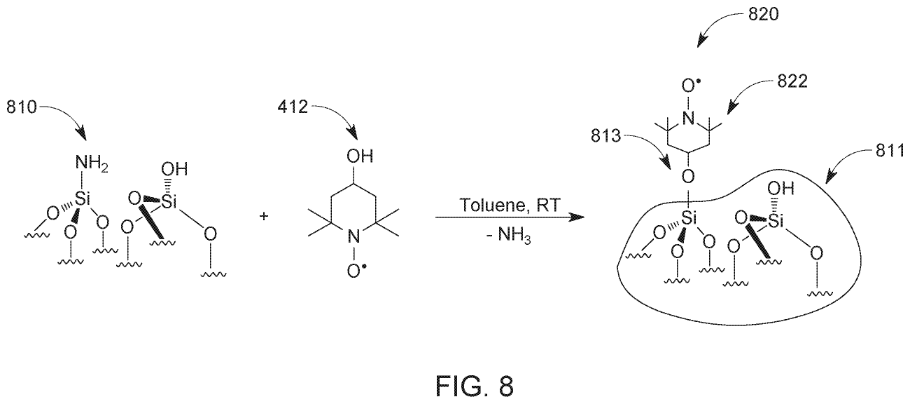

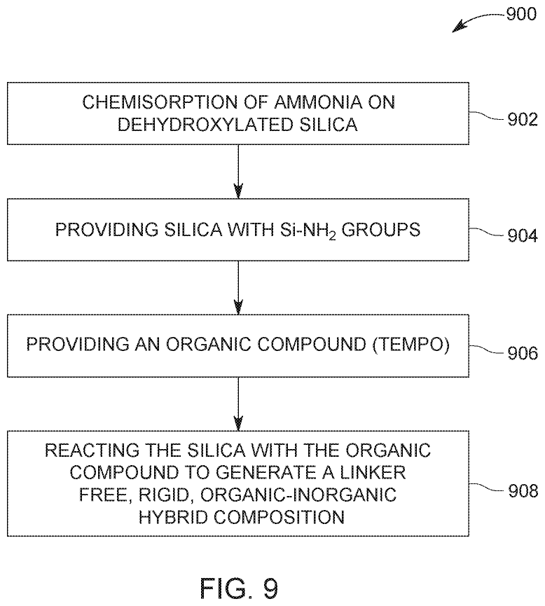

In one embodiment, a novel method is based on a single-step reaction between (1) silica having a silylamine (Si—NH₂) group and (2) 4-hydroxy-TEMPO. FIG. 8 schematically illustrates this method. The starting molecule 810, i.e., silica with Si—NH2 group, may be synthetized in step 902, as schematically illustrated in the flow chart of FIG. 9, by chemisorption of ammonia on dehydroxylated silica surface. This step is optional. If step 902 is missing, the method may include a step 904 of providing the silica with the Si—NH₂ groups 810. The method may further include a step 906 of providing an organic compound, in this case TEMPO 412. However, as discussed later, other organic compounds 1402 and 1910 may be used instead of the TEMPO 412. The silica with Si—NH₂ groups 810 then interacts in step 908 (this is the single step that is required for this method) with the TEMPO 412 at room temperature (RT), in the presence of toluene, and results in the formation of a linker free, rigid, TEMPO functionalized silica composition 820. Note that the TEMPO functionalized silica composition 820 includes a silica 811 stripped of an NH₂ group. The linker free TEMPO functionalized silica composition 820 is a TEMPO-2,2,6,6-tetramethyl-1-piperidinyloxy in this embodiment. The term “rigid” in this embodiment means that an interaction between adjacent TEMPO groups is prevented as the TEMPO groups cannot move enough relative to the silica base to interact with each other.

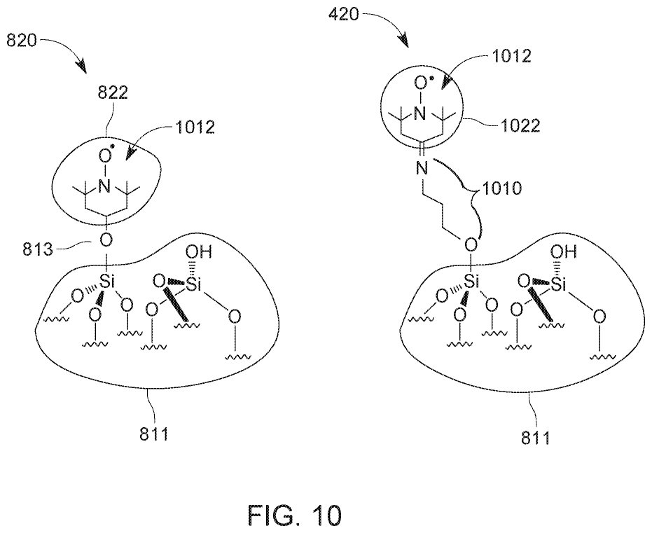

The rigidity of the composition 820 is a feature of the method illustrated in FIG. 9 and is due to the direct immobilization of the TEMPO group 822 (which corresponds to the TEMPO molecule 412 without the H at the hydroxyl OH) to the silica 811 stripped of the NH₂ group (called herein a silica 811 stripped of the amine group) surface. This is so because of the absence of a linker, as schematically illustrated in FIG. 10. The TEMPO molecule 412 is directly attached to an O atom 813 and the O atom 813 is directly attached to the silica 811 stripped of the NH₂ group. To the contrary, the catalysts discussed above, for example, SiliaCAT-TEMPO 420, also illustrated in FIG. 10, includes a linker group 1010 (which includes C and N atoms), that links a modified TEMPO group 1022 (which lacks the hydroxyl OH) to the silica 810's surface. In this embodiment, the “linker” is defined as a set of atoms. Because the composition 820 shows only one single atom 811 between the organic part 1012 and the inorganic part 811, it is considered to be a linker free composition. The absence of the linker for the new linker free TEMPO functionalized silica composition 820 minimizes the flexibility of the organic moiety 822 and so, the formation of hydrogen interactions, and steric hindrance between the TEMPO molecules. Thus, the nitroxyl group (N—O) 1012 availability is improved. The ammonia-NH₃ formed in the process illustrated in FIG. 8 can be recycled.

Comparing the process 900 for generating the linker free TEMPO functionalized silica composition 820 to the SiliaCAT-TEMPO catalyst 420 process, it is noted that the process 900 is a single-step (step 908 in FIG. 9 as step 902 is optional and steps 904 and 906 do not involve any chemical reaction) post-synthesis method while the process for the catalyst 420 is multi-step. For the process 900, there is no need to synthesize an organoalkoxysilane, while the catalyst 420 requires such a material. The process 900 uses only existing commercially available compounds while the process for making the catalyst 420 requires the modification of such commercially available compounds. Further, the process 900 uses fewer organic solvents and no coupling agent. The process of making the catalyst 420 requires other catalysts, coupling agents, solvents, etc. This means that the process 900 may be scaled up and is cost efficient.

In addition, the process 900 achieves a well-defined functionalization (i.e., single site strategy) while the process of making the catalyst 420 uses a random functionalization. Also, the process 900 results in a large availability of the nitroxyl groups, no linkers, no flexibility of the TEMPO groups relative to the silica's surface, an thus less electrostatic interactions between the various groups, which is contrary to the process of making the catalyst 420, where there is a limited availability of the nitroxyl groups, and due to the long alkyl linkers, there is flexibility of the TEMPO groups and thus electrostatic interactions between the groups, which is undesirable.

In one experiment performed by the inventors, the silica 810 having the silylamine surface group (0.014 mmol, 1 equivalent, also known as 1 eq.) was reacted (step 908 in FIG. 9) with 4-hydroxyl TEMPO 412 (2.5 eq., 0.035 mmol) in a double Schlenk flask using dry toluene (15 ml) as solvent in a glovebox. The reaction mixture was stirred at RT for 18 h under inert conditions. After this, it was washed multiple times with toluene and filtered. The resultant solid was dried on a high vacuum line (10⁻⁵ mbar) overnight. The resulting catalyst 820 was characterized by Fourier Transform Infrared Spectroscopy (FTIR) spectroscopy, electron paramagnetic resonance (EPR), and thermogravimetric analysis (TGA).

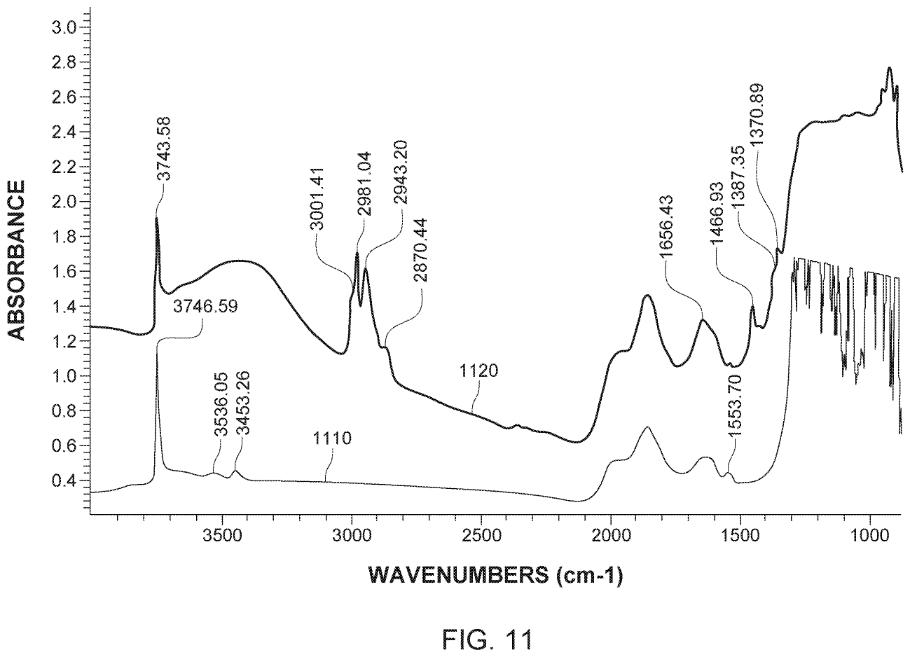

The FTIR spectrum (see FIG. 11) of the TEMPO-functionalized silica composition 820 shows that the typical peaks at 3536, 3453, and 1553 cm⁻¹ corresponding to the functional group Si—NH₂, visible on the curve 1110 and corresponding to the silica 810, have disappeared from the curve 1120, which corresponds to the composition 820. The same figure shows that new peaks appeared at 1656, 1466, 1387, and 1370 cm⁻¹, which were attributed to the presence of N—O, C—H, and C—N bonds, respectively, in the new composition 820. This evidence proved that the TEMPO moiety was successfully grafted to the silica support.

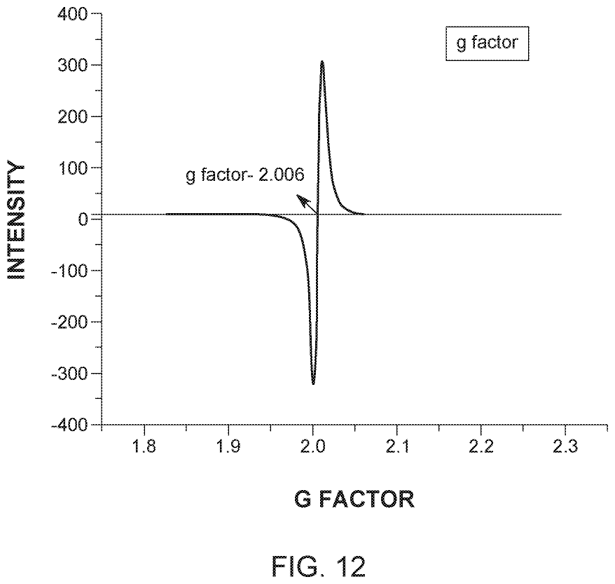

To confirm the presence of the radical TEMPO, EPR spectroscopy was performed on the TEMPO-functionalized silica composition 820. As shown in FIG. 12, the g-factor of the TEMPO-functionalized silica composition 820 was observed as 2.006, which corresponded to a typical N—O• radical on TEMPO. This result provides strong evidence that the silica functionalized by TEMPO groups (i.e., composition 820) has been successfully achieved.

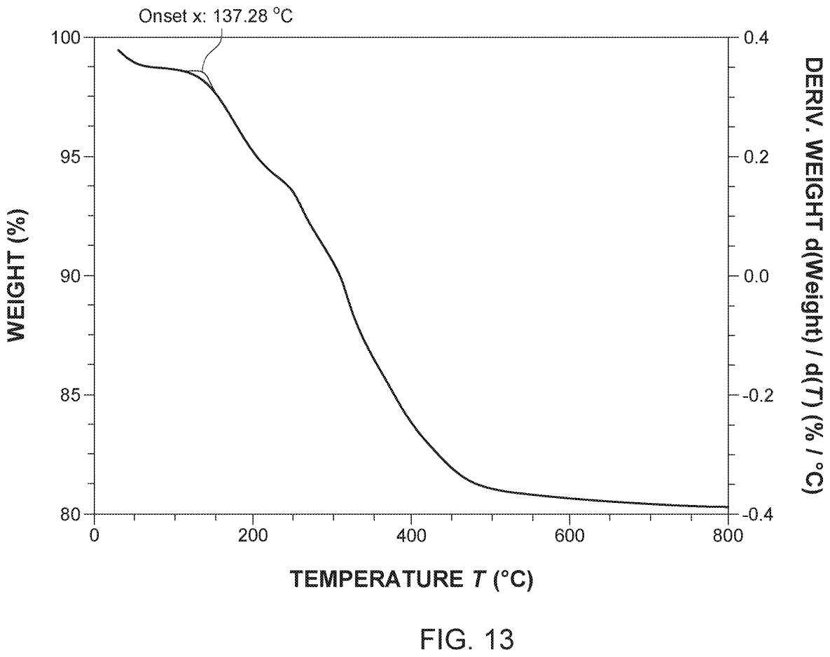

The thermal stability of the new composition 820 was determined using TGA analysis (which shows the mass of the composition, in percentage, relative to the initial mass, versus the temperature), which is presented in FIG. 13. The composition 820 was found to be stable up to 137 °C. Most likely, after this temperature, the organic part of the functionalized composition starts to decompose.

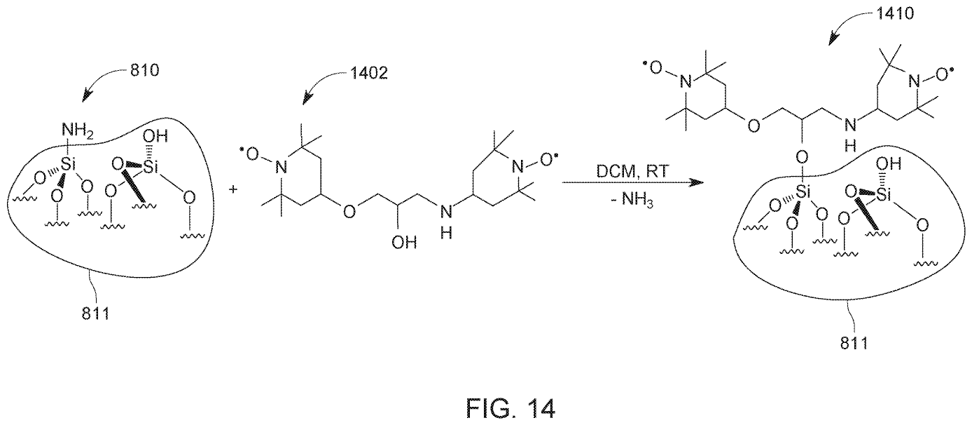

The method 900 may be expanded to generate TOTAPOL-functionalized silica, also called TOTAPOL-1-(TEMPO-4-oxy)-3-(TEMPO-4-amino)propan-2-ol, where TOTAPOL is a bi-radical nitroxyl compound. FIG. 14 schematically illustrates the one step reaction between the silica with Si—NH2 groups 810 and TOTAPOL 1402 at RT in the presence of DCM, which results in the formation of the TOTAPOL-1-(TEMPO-4-oxy)-3-(TEMPO-4-amino)propan-2-ol or TOTAPOL functionalized silica composition 1410. Note that the TOTAPOL functionalized silica composition 1410 is also linker free functionalized silica, similar (in the sense of the “linker free” feature) to the linker free TEMPO functionalized silica composition 820. Thus, both compositions 820 and 1410 are called in this document a linker free, rigid, functionalized silica composition. In one embodiment, the same procedure (e.g., step 908 in FIG. 9) may be applied to other types of radicals, for example, sulfoxyl, trityl, etc. as long there is a hydroxyl group.

In one experiment, the inventors reacted silica having silylamine surface groups 810 (0.0089 mmol, 1 eq.) with TOTAPOL 1402 (2.5 eq., 0.025 mmol) in a double Schlenk flask using dry dichloromethane (15 ml) as a solvent in a glovebox. The reaction mixture was stirred at RT for 18 h under inert conditions. After this, the resultant product was washed multiple times with DCM and filtered. The resultant solid, TOTAPOL functionalized silica composition 1410, was dried on a high vacuum line (10⁻⁵ mbar) overnight. The FTIR spectrum (not shown) of the TOTAPOL-functionalized silica composition 1410 shows that the typical peaks at 3536, 3453 and 1553 cm⁻¹ corresponding to the functional group Si—NH₂ disappeared, while new peaks appeared at 1655, 1467, 1421 and 1365, cm⁻¹, which were attributed to the presence of N—O, C—H, and C—N bonds, respectively. These results evidence that the TOTAPOL moiety was successfully grafted to the silica support.

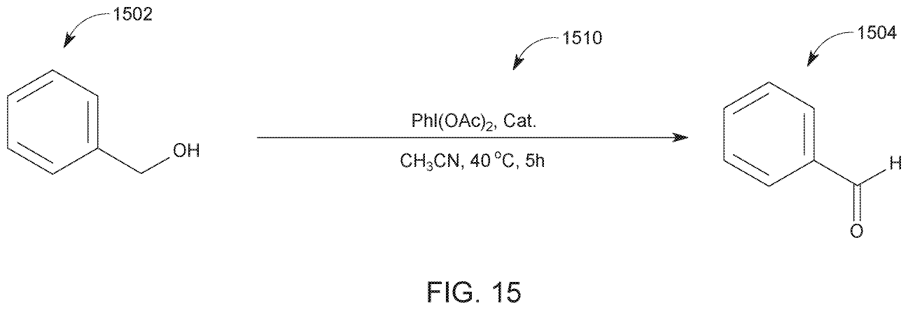

The linker free functionalized silica composition (which may be the linker free TEMPO functionalized silica composition 820 or the linker free TOTAPOL functionalized silica composition 1410) may be used in various fields, for example, oxidation reactions, materials for dynamic nuclear polarization (DNP), etc. For the oxidation reactions, the linker free functionalized silica composition 1510 (which may be either 1410 or 820) was used with benzyl alcohol 1502 in the presence of O₂ or other reagents such as PhI(OAc)₂ at 40 °C for about 5 h to generate benzaldehyde 1504, as schematically illustrated in FIG. 15. The composition 1510 showed similar performance with the existing catalysts used for this reaction.

In a different application, the linker free, rigid, functionalized silica composition 1510 was used as the material for DNP to improve the sensitivity in Nuclear Magnetic Resonance (NMR) spectroscopy experiments. DNP enhances the intensity of NMR signals and requires the use of nitroxyl radical (i.e., TEMPO, TOTAPOL etc.) as a source of polarization. The polarization is then transferred from the unpaired electrons of the nitroxyl radical to the nuclei (¹H, ¹³C, ²⁹Si, etc.) of interest of the analyzed materials to characterize them by in situ microwave (μwave) irradiation.

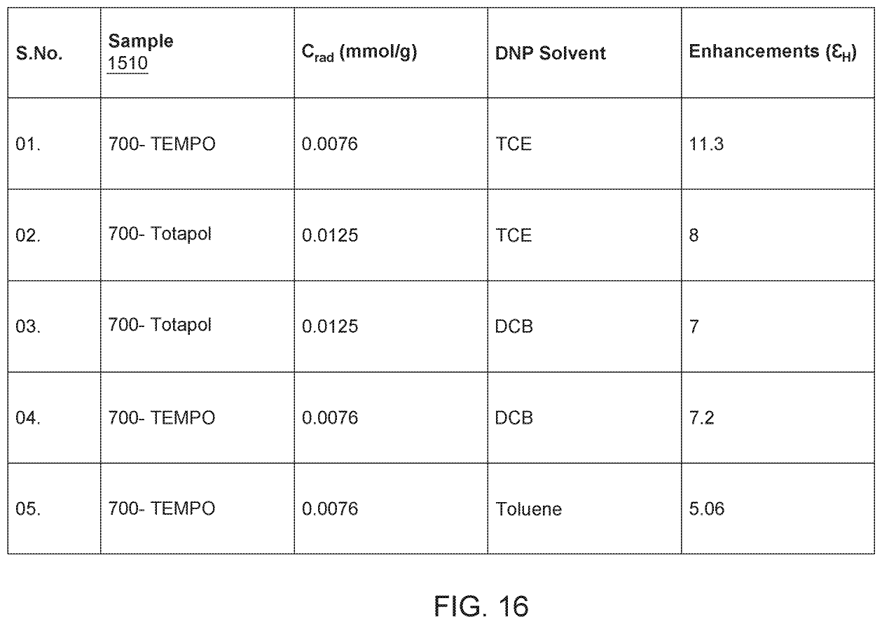

According to a first experiment, a spin counting has been performed for EPR. The spin counts of the radicals present in the TEMPO/TOTAPOL-functionalized silica composition 1510 were determined by continuous wave EPR spectroscopy. The solid materials were diluted 10 times with silica (just physical mixing) to avoid saturation of the spins and obtain reproducible results. In the experiment, the diluted solid sample was filled in Hirschmann glass capillaries, which were then closed with a cap. The spectra were recorded at room temperature and with a sweep width of 600 Gauss and attenuation between 26 and 14 dB. The amount of radical was determined by double integration of the CW spectra and number of spins were calculated by taking several parameters into account such as tube diameter, sample length, etc. The calculated values are summarized in the table in FIG. 16 (see column C_{rad} mmol/g). All the measured samples used either composition 820 or 1410, i.e., composition 1510.

The inventors also calculated the proton enhancement ξ_{H} obtained with TEMPO- and TOTAPOL-functionalized silica composition 1510. In the experiment, 8-10 mg of TEMPO- and TOTAPOL-functionalized silica was taken in a 3.2 mm sapphire NMR rotor and impregnated with solvent (TCE, DCB or Toluene—20 μl). After this, the tip of the rotor was blocked with a tight polytetrafluoroethylene (TEFLON) plug to prevent leakage of the solvent during spinning. The rotor was capped with a zirconia drive tip and inserted into the DNP spectrometer. The last column of the table in FIG. 16 shows the enhancement achieved by the catalyst 1510. A value over 7 indicates a good enhancement. The current system provides similar proton enhancement ξ_{H} with 10 times less radical concentration (0.0076 vs 0.088) indicating the homogeneity of the system.

The inventors performed a further experiment in which the proton enhancement ξ_{H} was obtained with TEMPO- and TOTAPOL-functionalized silica composition for the characterization of the analyte (e.g., urea or pyruvic acid). In the experiment, 8-10 mg of TEMPO-and TOTAPOL-functionalized silica composition were taken in a 3.2 mm sapphire NMR rotor and impregnated with solvent (TCE, DCB or Toluene—20 μl) and an analyte (10 μl, liquid analyte or 10 mg for solid analyte). The analyte used in the experiments was urea (solid) and pyruvic acid (liquid). After this, the tip of the rotor was blocked with a tight TEFLON plug to prevent leakage of solvent during spinning. The rotor was capped with a zirconia drive tip and inserted into the DNP spectrometer. In case of urea and TCE as solvent, the TEMPO functionalized silica (700—TEMPO) resulted in a proton enhancement ξ_{H}=16. This is the highest value obtained for these types of materials so far.

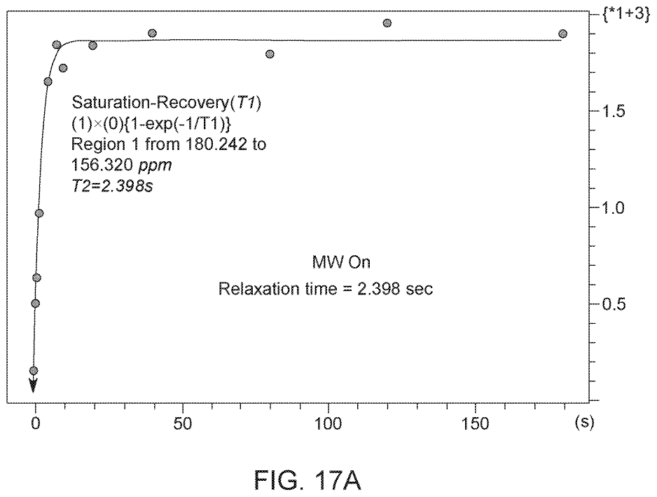

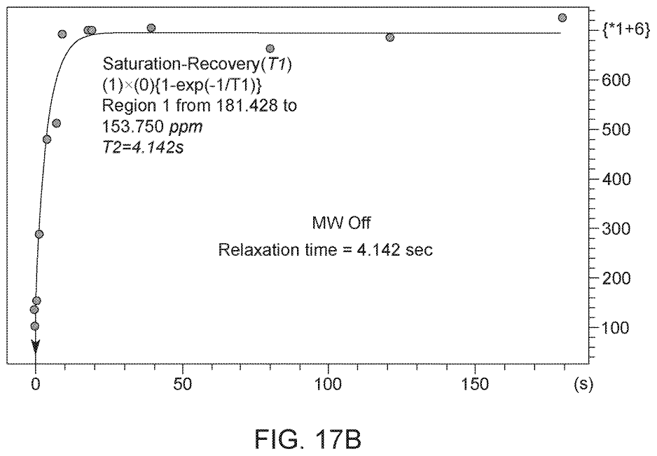

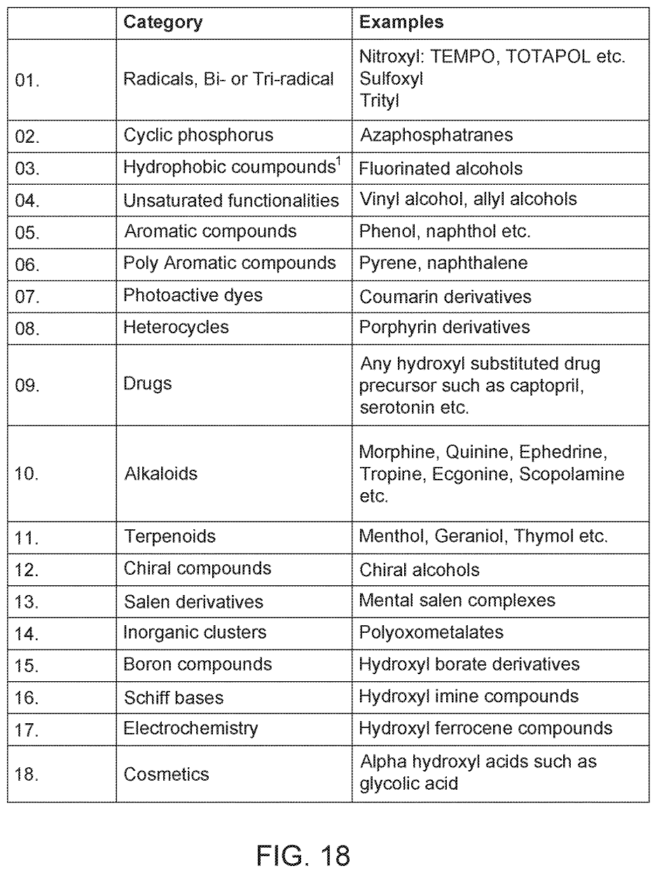

The inventors also performed DNP experiments with liquid analyte (Pyruvic acid) for studying the ¹³C relaxation time. In case of the pyruvic acid, the relaxation time of the carbonyl carbon was studied with the microwave (MW) on, see FIG. 17A, and MW off (see FIG. 17B). The polarization transfer from radically functionalized material leads to a decreased relaxation time (as observed in this case) of NMR active nuclei in DNP experiments. Lower relaxation time corresponds to faster analysis (low NMR measurement time) of complex molecules. In this case, the relaxation time was found to be ~2 times lower in DNP experimental conditions than the regular conditions. The figure plots the ¹³C relaxation time on the Y axis and the time on the X axis.

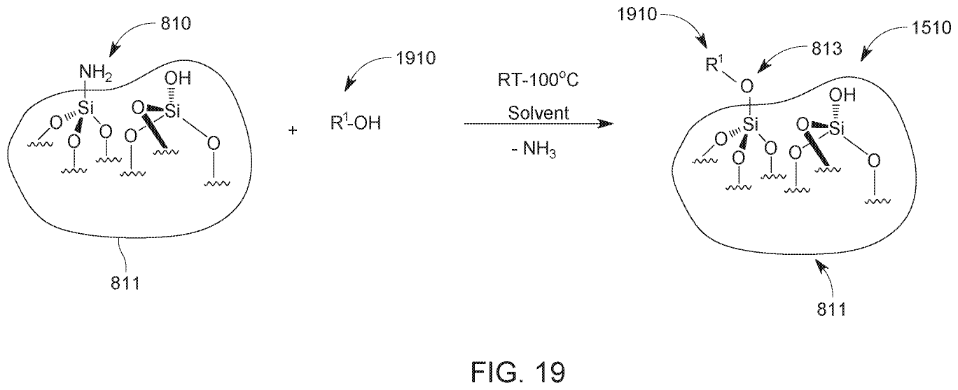

According to another embodiment, the step 908 of method 900 may be used to directly functionalize the silica 810 with one of many commercially available organic compounds having a functional group such as hydroxyl (OH). For example, any of the 19 organic classes of compounds from the table shown in FIG. 18 may be used to functionalize the silica 810. While FIG. 18 presents a list of 19 classes of organic compounds, this list is not exclusive. FIG. 19 shows the single-step 908 method of making a linker free, rigid, organic-inorganic hybrid composition (or material) 1510, which may include the compositions 820 or 1410 discussed above. Note that each of the compositions 820 and 1410 are linker free, which makes the molecule rigid, and also each of the composition 820 and 1410 include organic and inorganic materials, which results in the organic-inorganic hybrid material. The molecule is more rigid than the existing TEMPO-or TOTAPOL-functionalized catalysts because of the lack of the linker. Note that the linker confers the functionalized material a certain flexibility. The linker free, rigid, organic-inorganic hybrid composition 1510 shown in FIG. 19 is obtained by reacting the silica 810 with an organic compound 1910 (e.g., TEMPO, or TOTAPOL, or R¹—OH, where R¹ represents an alkyl or aryl group, a hydrocarbon chain or ring, or any of the organic compounds from the table of FIG. 18). The same reaction can be applied to other types of oxide supports, i.e., non-porous silica, mesoporous silica, microporous zeolite, mesoporous hierarchical zeolite, alumina, zirconia, ceria, titania, magnesium oxide, etc.

The hybrid compositions and associated methods discussed in this document are highly versatile in nature and applicable to many areas of chemical sciences. For example, on-demand functionalized materials can be manufactured depending on the targeting applications. Some of the targeted applications include heterogeneous catalysis, oxidation reactions, DNP applications, photochemistry, polymerization, biochemistry, sensing applications, radical chemistry, and biological application (MRI etc.)

The term “about” is used in this application to mean a variation of up to 20% of the parameter characterized by this term.

It will be understood that, although the terms first, second, etc. may be used herein to describe various elements, these elements should not be limited by these terms. These terms are only used to distinguish one element from another. For example, a first object or step could be termed a second object or step, and, similarly, a second object or step could be termed a first object or step, without departing from the scope of the present disclosure. The first object or step, and the second object or step, are both, objects or steps, respectively, but they are not to be considered the same object or step.

The terminology used in the description herein is for the purpose of describing particular embodiments and is not intended to be limiting. As used in this description and the appended claims, the singular forms “a,” “an” and “the” are intended to include the plural forms as well, unless the context clearly indicates otherwise. It will also be understood that the term “and/or” as used herein refers to and encompasses any possible combinations of one or more of the associated listed items. It will be further understood that the terms “includes,” “including,” “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Further, as used herein, the term “if” may be construed to mean “when” or “upon” or “in response to determining” or “in response to detecting,” depending on the context.

The disclosed embodiments provide a method for making an organically functionalized oxide composition without involving the use of environmentally damaging materials. It should be understood that this description is not intended to limit the invention. On the contrary, the embodiments are intended to cover alternatives, modifications and equivalents, which are included in the spirit and scope of the invention as defined by the appended claims. Further, in the detailed description of the embodiments, numerous specific details are set forth in order to provide a comprehensive understanding of the claimed invention. However, one skilled in the art would understand that various embodiments may be practiced without such specific details.

Although the features and elements of the present embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are in-tended to be within the scope of the claims.

The entire content of all the publications listed herein is incorporated by reference in this patent application.
[1] Kim, S.-B.; Lee, C.-H.; Jun, C.-H., Styrylsilane coupling reagents for immobilization of organic functional groups on silica and glass surfaces. Chem. Commun. 2018, 54(71 ), 9961-9964.
[2] Park, J.-W.; Kim, D.-S.; Kim, M.-S.; Choi, J.-H.; Jun, C.-H., A method for introducing organic functional groups on silica surfaces using a functionalized vinylsilane containing polymer. Polym. Chem. 2015, 6 (4), 555-560.

What is claimed is:

1. A linker free, rigid, organic-inorganic hybrid composition comprising:
a silica stripped of an NH₂ group;
an organic compound; and
a single oxygen atom directly linking the silica stripped of the NH₂ group to the organic compound.

2. The composition of claim 1, wherein the silica stripped of the NH₂ group is the inorganic part and the organic compound is the organic part.

3. The composition of claim 2, wherein there is no other chemical element connecting the inorganic part to the organic part.

4. The composition of claim 1, wherein there is no other chemical element connecting the silica stripped of the NH₂ group to the organic compound.

5. The composition of claim 1, wherein the organic compound is 2,2,6,6-tetramethylpiperidinyloxy.

6. The composition of claim 1, wherein the organic compound is a bi-nitroxyl radical.

7. The composition of claim 1, wherein the silica includes atoms of Si and O.

8. The composition of claim 7, wherein the silica further includes a hydroxyl group.

9. The composition of claim 1, wherein the organic compound includes sulfoxyl or trityl.

10. A method for making a linker free, rigid, organic-inorganic hybrid composition, the method comprising:
providing a silica having an NH₂ group;
providing an organic compound; and
reacting the silica with the NH₂ group with the organic compound so that a single oxygen atom directly links a silica stripped of the NH₂ group to the organic compound to form the linker free, rigid, organic-inorganic hybrid composition.

11. The method of claim 10, further comprising:
generating ammonia due to the reacting.

12. The method of claim 10, further comprising:
generating the silica having the NH₂ group from chemisorption of ammonia on dehydroxylated silica.

13. The method of claim 10, wherein the silica stripped of the NH₂ group is the inorganic part and the organic compound is the organic part.

14. The method of claim 13, wherein there is no other chemical element connecting the inorganic part to the organic part.

15. The method of claim 10, wherein there is no other chemical element connecting the silica stripped of the NH₂ group to the organic compound.

16. The method of claim 10, wherein the organic compound is 2,2,6,6-tetramethylpiperidinyloxy.

17. The method of claim 10, wherein the organic compound is a bi-nitroxyl radical.

18. The method of claim 10, wherein the silica includes atoms of Si and O and a hydroxyl group.

19. The method of claim 10, wherein the organic compound includes sulfoxyl or trityl.

20. The method of claim 10, wherein no organoalkoxysilane is used for forming the linker free, rigid, organic-inorganic hybrid composition.
